(19) United States
(12) Patent Application Publication
(10) Pub. No.: US 2024/0008849 A1
(43) Pub. Date: Jan. 11, 2024

(54) MEDICAL SYSTEM, METHOD FOR PROCESSING MEDICAL IMAGE, AND MEDICAL IMAGE PROCESSING APPARATUS

(71) Applicant: TERUMO KABUSHIKI KAISHA, Tokyo (JP)

(72) Inventors: Yuki SAKAGUCHI, Fujisawa Kanagawa (JP); Takanori TOMINAGA, Hadano Kanagawa (JP)

(21) Appl. No.: 18/471,251

(22) Filed: Sep. 20, 2023

Related U.S. Application Data
(63) Continuation of application No. PCT/JP2022/010150, filed on Mar. 9, 2022.

(30) Foreign Application Priority Data
Mar. 24, 2021 (JP) 2021-050688

(51) Int. Cl. A61B 8/00; G06V 10/70; A61B 8/12; A61B 8/08

(52) U.S. Cl. CPC A61B 8/463; G06V 10/70; A61B 8/12; A61B 8/0841; A61B 8/0891; G06V 2201/031; A61B 5/0066

(57) ABSTRACT

A medical system includes a catheter that includes a sensor and can be inserted into a luminal organ, a display apparatus, and an image processing apparatus configured to: store a plurality of pieces of support information each related to a medical operation or diagnosis on the organ and associated with a type of an object, generate an image of the organ based on a signal output from the sensor of the catheter, input the generated image to a machine learning model and acquire an output indicating a type of an object that is present in the image, acquire input information indicating a medical operation or diagnosis to be performed, determine one of the pieces of support information corresponding to the type of the object and the medical operation or diagnosis indicated by the input information, and cause the display apparatus to display said one of the pieces of support information.

### CROSS-REFERENCE TO RELATED APPLICATION

This application is a continuation of International Patent Application No. PCT/JP2022/010150 filed Mar. 9, 2022, which is based upon and claims the benefit of priority from Japanese Patent Application No. 2021-050688, filed on Mar. 24, 2021, the entire contents of which are incorporated herein by reference.

### FIELD

Embodiments described herein relate generally to a medical system, a method for processing a medical image of a luminal organ, and a medical image processing apparatus.

### BACKGROUND

An ultrasonic tomographic image of a blood vessel is generated by an intravascular ultrasound (IVUS) method using a catheter during an ultrasound examination of the blood vessel. Meanwhile, for the purpose of assisting diagnosis by a physician, a technology of adding information to a blood vessel image by image processing or machine learning has been developed. Such a technology includes a feature detection method for detecting a lumen wall, a stent, and the like included in the blood vessel image.

### SUMMARY OF THE INVENTION

Embodiments of the present disclosure provide a computer program or the like that provides useful information to an operator of a catheter about an object included in a medical image that is obtained by scanning a luminal organ with the catheter.

According to one embodiment, a medical system comprises a catheter that includes a sensor and can be inserted into a luminal organ; a display apparatus; and an image processing apparatus configured to: store a plurality of pieces of support information each related to a medical operation or diagnosis on the luminal organ and associated with a type of an object, generate an image of the luminal organ based on a signal output from the sensor of the catheter, input the generated image to a machine learning model and acquire an output indicating a type of an object that is present in the image, acquire input information indicating a medical operation or diagnosis to be performed, determine one of the pieces of support information corresponding to the type of the object and the medical operation or diagnosis indicated by the input information, and cause the display apparatus to display said one of the pieces of support information.

According to the present disclosure, it is possible to provide a system and the like that provides useful information to an operator of a catheter according to an object included in a medical image of a luminal organ obtained by scanning the luminal organ with the catheter.

### BRIEF DESCRIPTION OF THE DRAWINGS

FIG. 1 is a diagram illustrating a configuration example of an image diagnosis system.

FIG. 2 is a schematic diagram of an image diagnosis catheter.

FIG. 3 is an explanatory diagram illustrating a cross section of a blood vessel through which a sensor unit is inserted.

FIGS. 4A and 4B are explanatory diagrams of tomographic images.

FIG. 5 is a block diagram illustrating a configuration example of an image processing apparatus.

FIG. 6 is a diagram illustrating an example of a learning model.

FIG. 7 is a diagram illustrating an example of a relation table.

FIG. 8 is a flowchart of information processing performed by the image processing apparatus.

FIG. 9 is a flowchart of an information provision procedure of stent implant.

FIG. 10 is a diagram illustrating a display example of information specifying reference portions.

FIG. 11 is a diagram illustrating a display example of information regarding stent implant.

FIG. 12 is a flowchart of an information provision procedure of endpoint determination.

FIG. 13 is a flowchart of processing for MSA (minimum stent area) calculation.

FIG. 14 is a diagram illustrating a visualized display example of an expanded state in the vicinity of a stent implant portion;

FIG. 15 is a diagram illustrating a display example of information regarding a desired expansion diameter.

FIG. 16 is a diagram illustrating a display example of information regarding endpoint determination.

FIG. 17 is a diagram illustrating an example of a relation table in a second embodiment.

FIG. 18 is a diagram illustrating an example of a combination table.

FIG. 19 is a flowchart of information processing performed by the image processing apparatus.

### DETAILED DESCRIPTION

Hereinafter, embodiments of the present disclosure will be described in detail with reference to the drawings. In each of the following embodiments, a cardiac catheter treatment as an endovascular treatment will be described as an example, but a luminal organ to be subjected to a catheter treatment is not limited to a blood vessel, and may be other luminal organs such as a bile duct, a pancreatic duct, a bronchus, and an intestine.

### First Embodiment

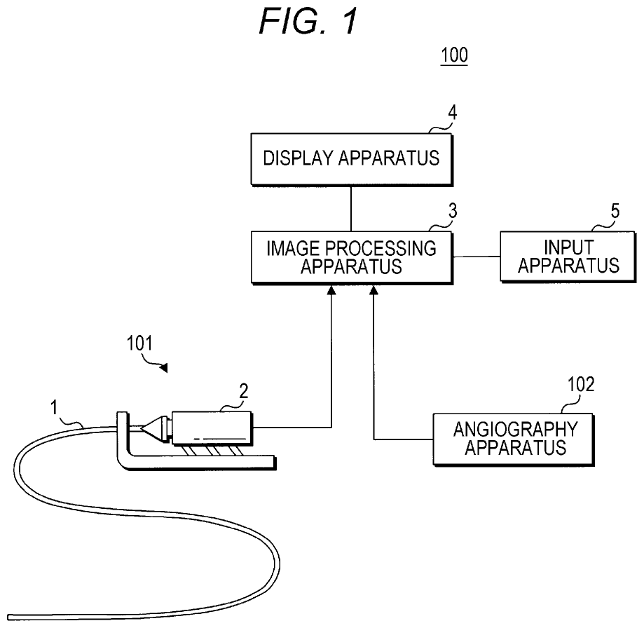

FIG. 1 is a diagram illustrating a configuration example of an image diagnosis system 100. In the present embodiment, the image diagnosis system 100 using a dual type catheter having functions of both intravascular ultrasound (IVUS) and optical coherence tomography (OCT) will be described. In the dual type catheter, a mode of acquiring an ultrasonic tomographic image only by IVUS, a mode of acquiring an optical coherence tomographic image only by OCT, and a mode of acquiring tomographic images by both IVUS and OCT are provided, and these modes can be switched and used. Hereinafter, an ultrasonic tomographic image and an optical coherence tomographic image are referred to as an IVUS image and an OCT image, respectively. In addition, an IVUS image and an OCT image are collectively referred to as tomographic images, and correspond to medical images.

The image diagnosis system 100 of the present embodiment includes an intravascular inspection apparatus 101, an angiography apparatus 102, an image processing apparatus 3, a display apparatus 4, and an input apparatus 5. The intravascular inspection apparatus 101 includes an image diagnosis catheter 1 and a motor drive unit (MDU) 2. The image diagnosis catheter 1 is connected to the image processing apparatus 3 via the MDU 2. The display apparatus 4 and the input apparatus 5 are connected to the image processing apparatus 3. The display apparatus 4 is, for example, a liquid crystal display (LCD), an organic EL (electro-luminescence) display, or the like, and the input apparatus 5 is, for example, a keyboard, a mouse, a trackball, a microphone, or the like. The display apparatus 4 and the input apparatus 5 may be integrated into a touch panel. Further, the input apparatus 5 and the image processing apparatus 3 may be integrated into one apparatus. Furthermore, the input apparatus 5 may be a sensor that receives a gesture input, a line-of-sight input, or the like.

The angiography apparatus 102 is connected to the image processing apparatus 3. The angiography apparatus 102 images a blood vessel from outside a living body of a patient using X-rays while injecting a contrast agent into the blood vessel of the patient to obtain an angiographic image that is a fluoroscopic image of the blood vessel. The angiography apparatus 102 includes an X-ray source and an X-ray sensor, and captures an X-ray fluoroscopic image of the patient by the X-ray sensor receiving X-rays emitted from the X-ray source. Note that the image diagnosis catheter 1 is provided with a radiopaque marker, and the position of the image diagnosis catheter 1 is visualized in the angiographic image using the marker. The angiography apparatus 102 outputs the angiographic image obtained by imaging to the image processing apparatus 3, and the angiographic image is displayed on the display apparatus 4 via the image processing apparatus 3. The display apparatus 4 displays the angiographic image and the tomographic image imaged using the image diagnosis catheter 1.

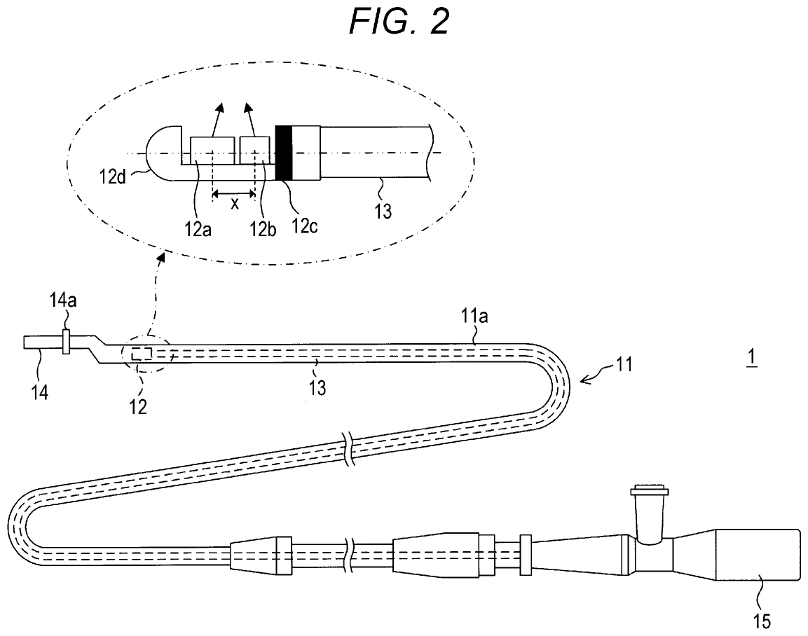

FIG. 2 is a schematic diagram of the image diagnosis catheter 1. Note that the region surrounded by a one-dot chain line on the upper side in FIG. 2 is an enlarged view of the region surrounded by a one-dot chain line on the lower side. The image diagnosis catheter 1 includes a probe 11 and a connector portion 15 disposed at an end of the probe 11. The probe 11 is connected to the MDU 2 via the connector portion 15. In the following description, a side far from the connector portion 15 of the image diagnosis catheter 1 will be referred to as a distal end side, and a side of the connector portion 15 will be referred to as a proximal end side. The probe 11 includes a catheter sheath 11a, and a guide wire insertion portion 14 through which a guide wire can be inserted is provided at a distal portion thereof. The guide wire insertion portion 14 constitutes a guide wire lumen, receives a guide wire previously inserted into a blood vessel, and guides the probe 11 to an affected part by the guide wire. The catheter sheath 11a forms a tube portion continuous from a connection portion with the guide wire insertion portion 14 to a connection portion with the connector portion 15. A shaft 13 is inserted into the catheter sheath 11a, and a sensor unit 12 is connected to a distal end side of the shaft 13.

The sensor unit 12 includes a housing 12d, and a distal end side of the housing 12d is formed in a hemispherical shape in order to suppress friction and catching with an inner surface of the catheter sheath 11a. In the housing 12d, an ultrasound transmitter and receiver 12a (hereinafter referred to as an IVUS sensor 12a) that transmits ultrasonic waves into a blood vessel and receives reflected waves from the blood vessel and an optical transmitter and receiver 12b (hereinafter referred to as an OCT (optical coherence tomographic) sensor 12b) that transmits near-infrared light into the blood vessel and receives reflected light from the inside of the blood vessel are disposed. In the example illustrated in FIG. 2, the IVUS sensor 12a is provided on the distal end side of the probe 11, the OCT sensor 12b is provided on the proximal end side thereof, and the IVUS sensor 12a and the OCT sensor 12b are arranged apart from each other by a distance X along the axial direction on the central axis of the shaft 13 between two chain lines in FIG. 2. In the image diagnosis catheter 1, the IVUS sensor 12a and the OCT sensor 12b are attached such that a radial direction of the shaft 13 that is approximately 90 degrees with respect to the axial direction of the shaft 13 is set as a transmission/reception direction of an ultrasonic wave or near-infrared light. Note that the IVUS sensor 12a and the OCT sensor 12b are desirably attached slightly shifted from the radial direction so as not to receive a reflected wave or reflected light on the inner surface of the catheter sheath 11a. In the present embodiment, for example, as indicated by the arrows on the upper side of FIG. 2, the IVUS sensor 12a is attached with a direction inclined to the proximal end side with respect to a radial direction as an irradiation direction of the ultrasonic wave, and the OCT sensor 12b is attached with a direction inclined to the distal end side with respect to the radial direction as an irradiation direction of the near-infrared light.

An electric signal cable (not illustrated) connected to the IVUS sensor 12a and an optical fiber cable (not illustrated) connected to the OCT sensor 12b are inserted into the shaft 13. The probe 11 is inserted into the blood vessel from the distal end side. The sensor unit 12 and the shaft 13 can move forward or rearward inside the catheter sheath 11a and can rotate in a circumferential direction. The sensor unit 12 and the shaft 13 rotate about the central axis of the shaft 13 as a rotation axis. In the image diagnosis system 100, by using an imaging core including the sensor unit 12 and the shaft 13, a state of the blood vessel is observed by an ultrasonic tomographic image captured from the inside of the blood vessel or an optical coherence tomographic image captured from the inside of the blood vessel.

The MDU 2 is a drive device to which the probe 11 of the image diagnosis catheter 1 is detachably attached by the connector portion 15, and controls the operation of the image diagnosis catheter 1 inserted into the blood vessel by driving a built-in motor according to an operation by a medical worker. For example, the MDU 2 performs a pull-back operation of rotating the sensor unit 12 and the shaft 13 inserted into the probe 11 in the circumferential direction while pulling the sensor unit 12 and the shaft 13 toward the MDU 2 side at a constant speed. The sensor unit 12 continuously scans the inside of the blood vessel at predetermined time intervals while moving from the distal end side to the proximal end side by the pull-back operation and continuously captures a plurality of transverse tomographic images substantially perpendicular to the probe 11 at predetermined intervals. The MDU 2 outputs reflected wave data of an ultrasonic wave received by the IVUS sensor 12a and reflected light data received by the OCT sensor 12b to the image processing apparatus 3.

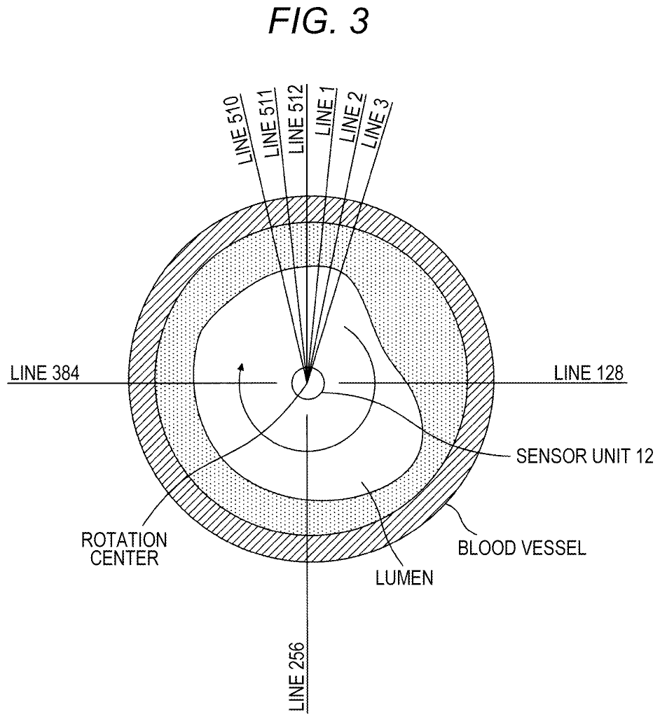

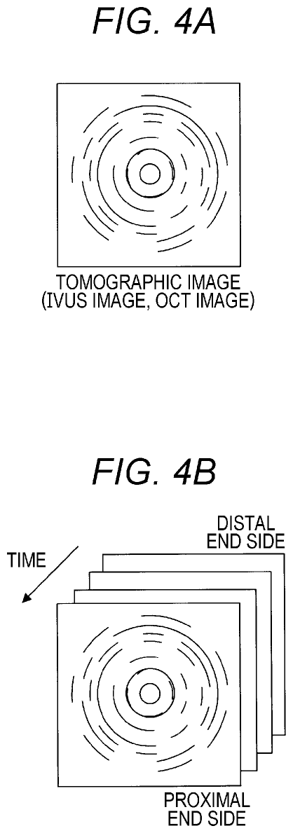

The image processing apparatus 3 acquires a signal data set which is the reflected wave data of the ultrasonic wave received by the IVUS sensor 12a and a signal data set which is reflected light data received by the OCT sensor 12b via the MDU 2. The image processing apparatus 3 generates ultrasound line data from the ultrasound signal data set, and generates an ultrasonic tomographic image of a transverse section of the blood vessel based on the generated ultrasound line data. In addition, the image processing apparatus 3 generates optical line data from the signal data set of the reflected light, and generates an optical tomographic image of a transverse section of the blood vessel based on the generated optical line data. Here, the signal data set acquired by the IVUS sensor 12a and the OCT sensor 12b and the tomographic image generated from the signal data set will be described. FIG. 3 is an explanatory diagram illustrating a cross section of the blood vessel through which the sensor unit 12 is inserted, and FIGS. 4A and 4B are explanatory diagrams of the tomographic images.

First, with reference to FIG. 3, operations of the IVUS sensor 12a and the OCT sensor 12b in the blood vessel, and signal data sets (i.e., ultrasonic line data and optical line data) acquired by the IVUS sensor 12a and the OCT sensor 12b will be described. When the imaging of the tomographic image is started in a state where the imaging core is inserted into the blood vessel, the imaging core rotates about the central axis of the shaft 13 as indicated by the arrow in FIG. 3. At this time, the IVUS sensor 12a transmits and receives an ultrasonic wave at each rotation angle. Lines 1, 2, . . . 512 indicate transmission/reception directions of ultrasonic waves at each rotation angle. In the present embodiment, the IVUS sensor 12a intermittently transmits and receives ultrasonic waves 512 times while rotating 360 degrees (i.e., one rotation) in the blood vessel. Since the IVUS sensor 12a acquires data of one line in the transmission/reception direction by transmitting and receiving an ultrasonic wave once, it is possible to obtain 512 pieces of ultrasonic line data radially extending from the rotation center during one rotation. The 512 pieces of ultrasonic line data are dense in the vicinity of the rotation center, but become sparse with distance from the rotation center. Therefore, the image processing apparatus 3 can generate a two-dimensional ultrasonic tomographic image as illustrated in FIG. 4A by generating pixels in an empty space of each line by known interpolation processing.

Similarly, the OCT sensor 12b also transmits and receives the measurement light at each rotation angle. Since the OCT sensor 12b also transmits and receives the measurement light 512 times while rotating 360 degrees in the blood vessel, it is possible to obtain 512 pieces of optical line data radially extending from the rotation center during one rotation. Moreover, for the optical line data, the image processing apparatus 3 can generate a two-dimensional optical coherence tomographic image similar to the IVUS image illustrated in FIG. 4A by generating pixels in a vacant space of each line by known interpolation processing. That is, the image processing apparatus 3 generates optical line data based on interference light generated by causing reflected light and, for example, reference light obtained by separating light from a light source in the image processing apparatus 3 to interfere with each other, and generates an optical tomographic image of the transverse section of the blood vessel based on the generated optical line data.

The two-dimensional tomographic image generated from the 512 pieces of line data in this manner is referred to as an IVUS image or an OCT image of one frame. Since the sensor unit 12 scans while moving in the blood vessel, an IVUS image or an OCT image of one frame is acquired at each position rotated once within a movement range. That is, since the IVUS image or the OCT image of one frame is acquired at each position from the distal end side to the proximal end side of the probe 11 in the movement range, as illustrated in FIG. 4B, the IVUS image or the OCT image of a plurality of frames is acquired within the movement range.

The image diagnosis catheter 1 has a radiopaque marker in order to confirm a positional relationship between the IVUS image obtained by the IVUS sensor 12a or the OCT image obtained by the OCT sensor 12b and the angiographic image obtained by the angiography apparatus 102. In the example illustrated in FIG. 2, a marker 14a is provided at the distal portion of the catheter sheath 11a, for example, the guide wire insertion portion 14, and a marker 12c is provided on the shaft 13 side of the sensor unit 12. When the image diagnosis catheter 1 configured as described above is imaged with X-rays, an angiographic image in which the markers 14a and 12c are visualized is obtained. The positions at which the markers 14a and 12c are provided are an example, the marker 12c may be provided on the shaft 13 instead of the sensor unit 12, and the marker 14a may be provided at a portion other than the distal portion of the catheter sheath 11a.

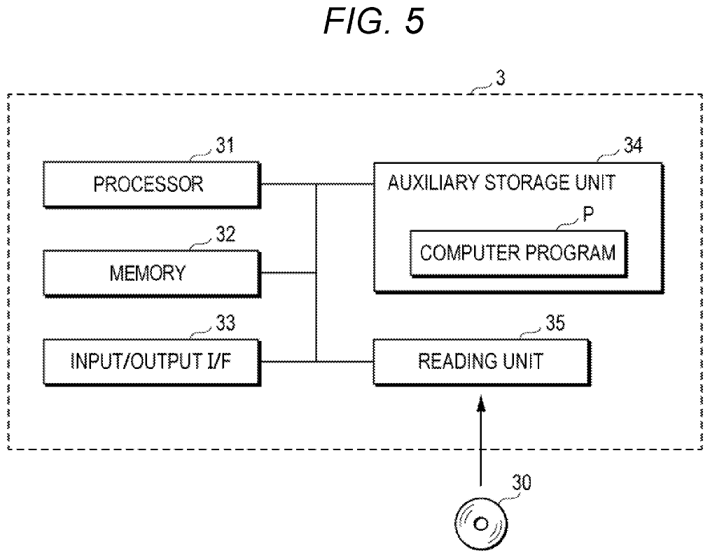

FIG. 5 is a block diagram illustrating a configuration example of the image processing apparatus 3. The image processing apparatus 3 includes a processor 31, a memory 32, an input/output interface (I/F) 33, an auxiliary storage unit 34, and a reading unit 35.

The processor 31 includes, for example, one or more central processing units (CPU), one or more micro-processing units (MPU), one or more graphics processing units (GPU), one or more general-purpose graphics processing units (GPGPU), and one or more tensor processing units (TPU). The processor 31 is connected to each hardware component of the image processing apparatus 3 via a bus.

The memory 32 includes, for example, a static random access memory (SRAM), a dynamic random access memory (DRAM), or a flash memory, and temporarily stores data necessary for the processor 31 to execute arithmetic processing.

The input/output I/F 33 is an interface circuit to which the intravascular inspection apparatus 101, the angiography apparatus 102, the display apparatus 4, and the input apparatus 5 are connected. The processor 31 acquires the IVUS image and the OCT image from the intravascular inspection apparatus 101 via the input/output I/F 33, and acquires the angiographic image from the angiography apparatus 102. In addition, the processor 31 controls the input/output I/F 33 to output medical image signals of the IVUS image, the OCT image, or the angiographic image to the display apparatus 4, thereby displaying the medical image on the display apparatus 4. Furthermore, the processor 31 acquires information input to the input apparatus 5 via the input/output I/F 33.

For example, a communication unit including a wireless communication device supporting 4G, 5G, or Wi-Fi may be connected to the input/output I/F 33, and the image processing apparatus 3 may be communicably connected to an external server such as a cloud server connected to an external network such as the Internet via the communication unit. The image processing apparatus 3 may communicate with the external server via the communication unit and the external network, refer to medical data, paper information, and the like stored in a storage device included in the external server, and perform processing for providing support information. Alternatively, the processor 31 may cooperatively perform the processing in the present embodiment by performing, for example, inter-process communication with the external server.

The auxiliary storage unit 34 is a storage device such as a hard disk, an electrically erasable programmable ROM (EEPROM), or a flash memory. The auxiliary storage unit 34 stores a computer program P executed by the processor 31 and various data necessary for processing by the processor 31. Note that the auxiliary storage unit 34 may be an external storage device connected to the image processing apparatus 3. The computer program P may be stored in the auxiliary storage unit 34 at the manufacturing stage of the image processing apparatus 3, or the computer program distributed by a remote server device may be acquired by the image processing apparatus 3 through communication and stored in the auxiliary storage unit 34. The computer program P may be recorded in a non-transitory computer readable recording medium 30 such as a magnetic disk, an optical disk, or a semiconductor memory, and the reading unit 35 may read the computer program P from the recording medium 30 and store the computer program P in the auxiliary storage unit 34.

The image processing apparatus 3 may be composed of multiple processing devices. In addition, the image processing apparatus 3 may be a server client system, a cloud server, or a virtual machine operating as software. In the following description, it is assumed that the image processing apparatus 3 is one processing device. In the present embodiment, the image processing apparatus 3 is connected to the angiography apparatus 102 that images two-dimensional angiographic images. However, the present invention is not limited to that configuration, and the image processing apparatus 3 may be connected to any apparatus that images a luminal organ of a patient and the image diagnosis catheter 1 from a plurality of directions outside the living body.

In the image processing apparatus 3 of the present embodiment, the processor 31 reads and executes the computer program P stored in the auxiliary storage unit 34, thereby executing processing of generating the IVUS image based on the signal data set received from the IVUS sensor 12a and the OCT image based on the signal data set received from the OCT sensor 12b. Note that, since observation positions of the IVUS sensor 12a and the OCT sensor 12b are shifted at the same imaging timing as described later, the processor 31 executes processing of correcting the shift of the observation positions in the IVUS image and the OCT image. Therefore, the image processing apparatus 3 of the present embodiment provides an image that is easy to read by providing the IVUS image and the OCT image in which the observation positions are matched.

In the present embodiment, the image diagnosis catheter is a dual type catheter having functions of both intravascular ultrasound and optical coherence tomography, but is not limited thereto. The image diagnosis catheter may be a single type catheter having the function of either the intravascular ultrasound or the optical coherence tomography. Hereinafter, in the present embodiment, the image diagnosis catheter has the function of the intravascular ultrasound, and will be described based on the IVUS image generated by the IVUS function. However, in the description of the present embodiment, the medical image is not limited to the IVUS image, and the processing of the present embodiment may be performed using the OCT image as the medical image.

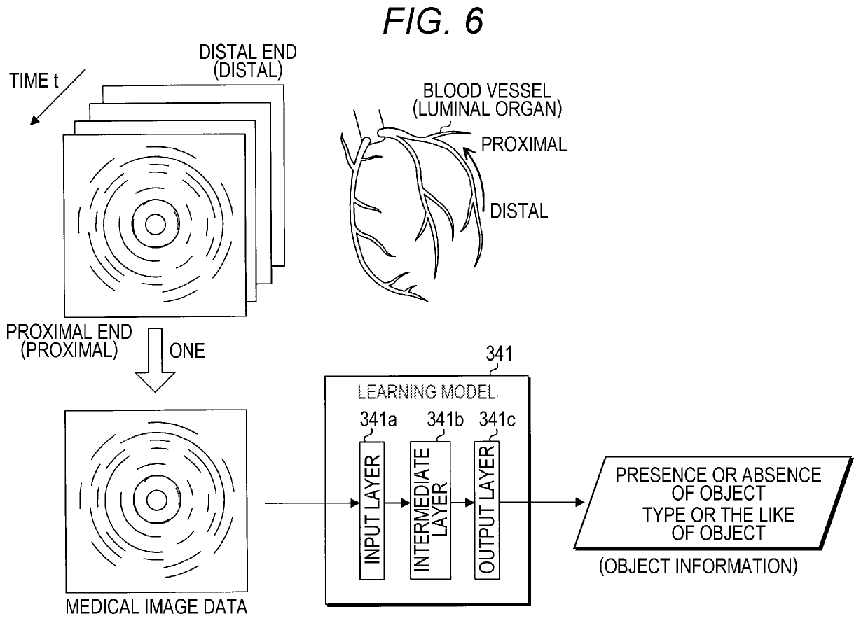

FIG. 6 is a diagram illustrating an example of a learning model 341. The learning model 341 is, for example, a neural network that performs object detection, semantic segmentation, or instance segmentation. Based on each IVUS image in the input IVUS image group, the learning model 341 outputs whether an object such as a stent or a plaque is included (i.e., present or absent) in the IVUS image, and in a case where the object is included (i.e., present), the learning model outputs a type or a class of the object, a region in the IVUS image, and estimation accuracy or a score.

The learning model 341 includes, for example, a trained convolutional neural network (CNN) by deep learning. The learning model 341 includes, for example, an input layer 341a to which a medical image such as an IVUS image is input, an intermediate layer 341b that extracts a feature amount of the image, and an output layer 341c that outputs information indicating a position and a type of an object included in the medical image. The input layer 341a of the learning model 341 has a plurality of neurons that receives an input of a pixel value of each pixel included in the medical image, and passes the input pixel value to the intermediate layer 341b. The intermediate layer 341b has a configuration in which a convolution layer for convoluting the pixel value of each pixel input to the input layer 341a and a pooling layer for mapping the pixel value convoluted by the convolution layer are alternately connected, and extracts the feature amount of an image while compressing pixel information of the medical image. The intermediate layer 341b passes the extracted feature amount to the output layer 341c. The output layer 341c includes one or a plurality of neurons that output the position, range, type, and the like of the image region of the object included in the image. Although the learning model 341 is the CNN, the configuration of the learning model 341 is not limited to the CNN. The learning model 341 may be, for example, a trained model having a configuration such as a neural network other than the CNN, a support vector machine (SVM), a Bayesian network, or a regression tree. Alternatively, the learning model 341 may input the image feature amount output from the intermediate layer to a support vector machine (SVM) to perform object recognition.

The learning model 341 can be generated by preparing training data in which a medical image including an object such as an epicardium, a side branch, a vein, a guide wire, a stent, a plaque deviating into a stent, a lipid plaque, a fibrous plaque, a calcified portion, blood vessel dissociation, thrombus, and haematoma and a label indicating a position or a region and a type of each object are associated with each other and causing an untrained neural network to perform machine training using the training data. According to the learning model 341 configured in this manner, by inputting the medical image such as the IVUS image to the learning model 341, information indicating the position and type of the object included in the medical image can be obtained. In a case where no object is included in the medical image, the information indicating the position and the type is not output from the learning model 341. Therefore, by using the learning model 341, the processor 31 can acquire whether the object is included (i.e., presence or absence) in the medical image input to the learning model 341, and in a case where the object is included, the control unit can acquire the type or class, the position (region in the medical image), and the estimation accuracy or score of the object.

The processor 31 generates object information regarding the presence or absence and the type of the object included in the IVUS image based on the information acquired from the learning model 341. Alternatively, the processor 31 may use the information output from the learning model 341 as the object information.

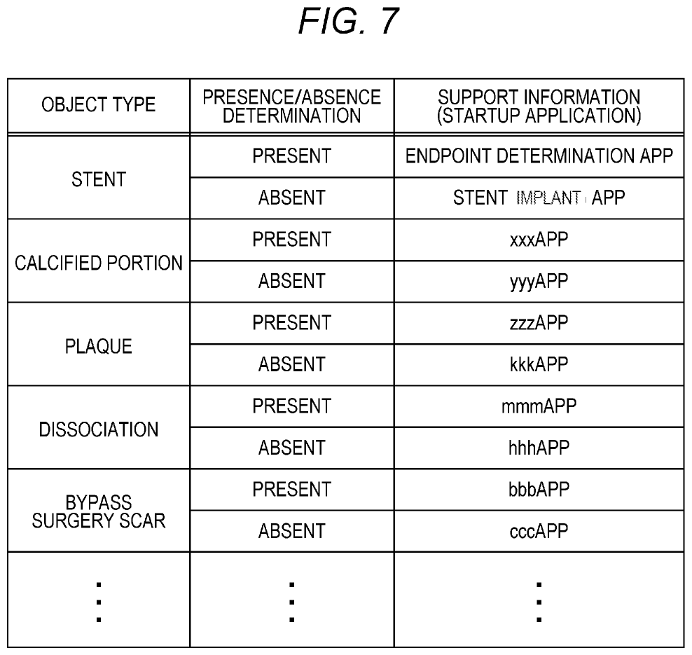

FIG. 7 is a diagram illustrating an example of a relation table. The auxiliary storage unit 34 of the image processing apparatus 3 stores the type of each object and the support information in association with each other, for example, as a relation table. The relation table includes, for example, an object type, presence/absence determination, and support information (startup application) as management items or fields of the table.

In the management item of the object type, for example, the type of the object such as the stent, calcified portion, plaque, blood vessel dissociation, and bypass surgery scar is stored. In the management item of the presence/absence determination, the presence/absence in each type of each object is stored. In the management item of the support information (startup application), the content of the support information according to the presence or absence of the object type stored in the same record or the application name for providing the support information is stored.

The processor 31 compares the relation table stored in the storage unit with the object information generated using the learning model 341, thereby efficiently determining the support information (startup application) according to the object information. For example, in a case where the object information relates to the stent and the object information indicates presence of the stent, the processor 31 performs provision processing (i.e., execution of an endpoint determination application (APP)) for providing support information regarding endpoint determination for determining the effectiveness or safety of the procedure, for example. In a case where the object information indicates absence of the stent, the processor 31 performs provision processing (i.e., execution of a stent implant APP) for providing support information regarding stent implant.

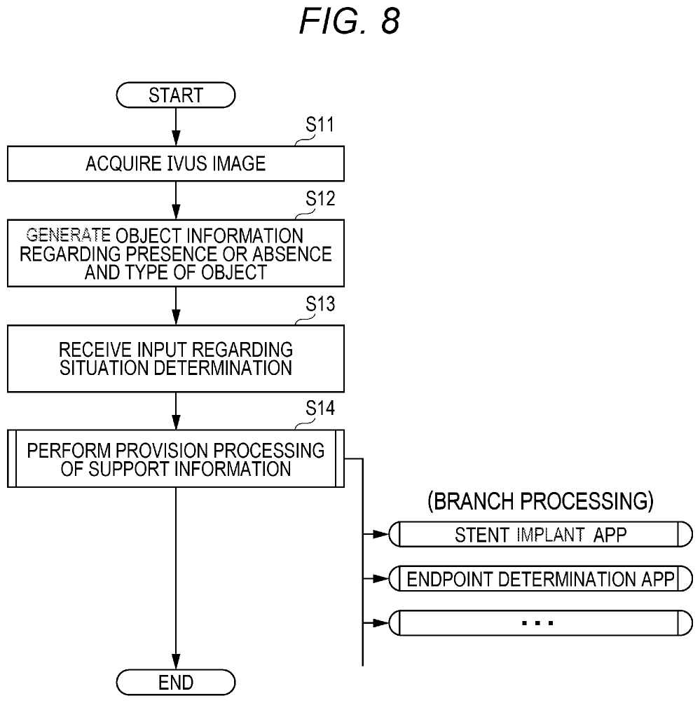

FIG. 8 is a flowchart of information processing performed by the processor 31. The processor 31 of the image processing apparatus 3 executes the following processing based on input data or the like output from the input apparatus 5 in response to an operation of an operator of the image diagnosis catheter 1 such as a physician.

The processor 31 acquires the IVUS image (S11). The processor 31 reads the IVUS image group obtained by pull-back, thereby acquiring a medical image including these IVUS images.

The processor 31 generates object information regarding the presence or absence and the type of the object included in the IVUS image (S12). For example, the processor 31 inputs the acquired IVUS image group to the learning model 341, and generates the object information based on the presence or absence and the type of the object estimated by the learning model 341. The learning model 341 includes, for example, a neural network that performs object detection, semantic segmentation, or instance segmentation, and the learning model 341 outputs, based on each IVUS image in the input IVUS image group, whether the object such as the stent or the plaque is included (i.e., presence or absence) in the IVUS image, and in a case where the object is included (i.e., presence), the type or class of the object, the region in the IVUS image, and the estimation accuracy or score.

The processor 31 generates the object information on the IVUS image using the estimation result (i.e., the presence or absence and the type of the object) output from the learning model 341. As a result, the object information indicates the presence or absence and the type of the object included in the IVUS image that is the original data of the object information. The object information may be generated as, for example, an XML format file, and the presence or absence of each type of individual object may be added or tagged to all types of objects to be estimated by the learning model 341. As a result, for example, the processor 31 can determine whether the stent is included (i.e., presence or absence) in the IVUS image, that is, whether the stent is implanted in the blood vessel. In the present embodiment, the processor 31 generates the object information on the IVUS image using the learning model 341, but the present invention is not limited thereto, and the processor 31 may determine the presence or absence and the type of the object included in the IVUS image using an image analysis means by edge detection, pattern matching, or the like for the IVUS image, and generate the object information using the determination result.

The processor 31 determines that an input regarding situation determination by the operator is received (S13). The input regarding situation determination includes one of surgery progress, a medical condition, or the like made by an operator of the image diagnosis catheter 1 such as a physician. The processor 31 determines the support information to be provided based on the object information or the like, and performs provision processing of the support information (S14). The processor 31 determines the support information to be provided based on the generated object information and the received information regarding the situation determination, and performs the provision processing of the support information.

For example, in a case where the input situation determination relates to a stent, the processor 31 determines the presence or absence of the stent, that is, whether it is before or after implant of the stent in the object information generated based on the IVUS image, and determines to provide the support information according to the determination result. The provision of the support information includes a provision mode in which the support information itself is superimposed and displayed on the screen of the display apparatus 4, and execution of an application that executes calculation processing or the like for generating and presenting the support information.

Before the implant of the stent, the processor 31 determines support information regarding the stent implant as the support information, and activates the stent implant APP for assisting determination of stent size and prediction of complications, for example. After implant of the stent, the processor 31 determines the support information regarding the endpoint determination as the support information to be provided, and activates, for example, the endpoint determination APP for assisting the endpoint determination and assisting the prediction of complications. The processor 31 may refer to the relation table stored in the auxiliary storage unit 34 and determine the support information according to the presence or absence of each type of individual object included in the object information.

In the illustration in the present embodiment, a flow of processing regarding provision of each piece of support information in a case where the presence or absence and the type of the object indicated by the object information are the presence or absence of the stent (after the implant, before the implant) will be described as an example. The flow is an example, and the processor 31 performs provision processing (i.e., execution of the application) for providing support information defined in advance according to the presence or absence of individual types of objects exemplified in the relation table, such as the presence or absence of the calcified portion, the presence or absence of the plaque, the presence or absence of the blood vessel dissociation, and the presence or absence of the bypass surgery scar. In performing the provision processing for providing the support information according to the presence or absence and the type of the object indicated by the object information as described above, the branch processing according to the presence or absence and the type of the object may be performed using, for example, a case sentence in the program executed by the processor 31.

In the example of the relation table, the support information defined according to the presence or absence of each type of object is not limited to the single case, and a plurality of pieces of support information may be defined. In this case, the provision processing may be performed on all of the plurality of pieces of support information, or the names and the like of the plurality of pieces of support information may be displayed on the display apparatus 4 in the form of a list, and the selection of one of the pieces of support information may be accepted to perform the provision processing of the selected support information.

In the present embodiment, the processor 31 determines the support information based on the object information and the information regarding the situation determination, but the present invention is not limited thereto, and the processor 31 may determine the support information based on only the object information. That is, the processing of receiving an input related to the situation determination by the operator may be unnecessary, the support information to be provided may be determined based on only the object information generated based on the IVUS image, and the provision processing such as activation of an application for providing the support information may be performed.

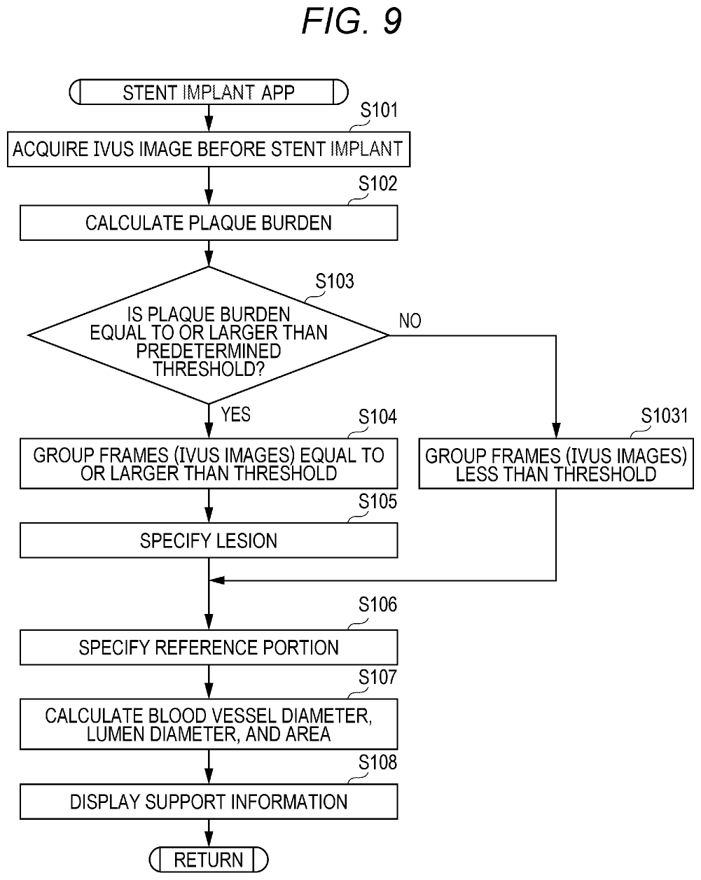

FIG. 9 is a flowchart illustrating an information provision procedure of stent implant. In this flow, the provision processing for starting stent implant APP to provide the support information regarding stent implant will be described with reference to FIG. 9.

The processor 31 acquires IVUS images before stent implant (S101). The processor 31 acquires a plurality of IVUS images before stent implant corresponding to one pull back. The IVUS image may be used for generating object information. When a plurality of IVUS images (hereinafter referred to as an IVUS image group) is used in generating the object information, any IVUS image included in the IVUS image group may be acquired.

The processor 31 calculates a plaque burden (S102). For example, the processor 31 segments the lumen and the blood vessel shown in the acquired IVUS image using the learning model 341, and calculates the plaque burden. These cross-sectional areas in the tomographic view may be calculated by segmenting the lumen and the blood vessel, and the area of the plaque burden or the plaque may be calculated by dividing or subtracting the area of the blood vessel and the area of the lumen.

The processor 31 determines whether the plaque burden is equal to or larger than a predetermined threshold (S103). The processor 31 determines whether the plaque burden is equal to or larger than a predetermined threshold, thereby classifying the plaque burden based on the threshold. The processor 31 classifies the calculated plaque burden area based on a predetermined threshold such as 40%, 50%, or 60%, for example. The threshold may be configured to enable a plurality of settings.

When the plaque burden is equal to or larger than the predetermined threshold (YES in S103), the processor 31 groups the frames of the IVUS images equal to or larger than the threshold (S104). The processor 31 groups frames equal to or larger than the plaque burden threshold as a lesion. In a case where the lesion portions are scattered apart from each other, the lesion portions may be grouped (L1, L2, L3 . . . ). However, when the interval or distance between the groups is 0.1 to 3 mm or less, the groups may be the same.

The processor 31 specifies a group including the maximum value of plaque burden as a lesion (S105). The processor 31 specifies a group including a site where the maximum value of plaque burden, that is, the lumen diameter becomes the minimum value, as a lesion.

If the difference is not the predetermined threshold or more (NO in S103), that is, if the difference is less than the predetermined threshold, the processor 31 groups the frames less than the threshold (S1031). In a case where the value is less than the predetermined threshold, the processor 31 groups frames less than the threshold as a reference portion. In a case where the plaque burden to be the reference portion is scattered apart, the plaque burden may be grouped (R1, R2, R3, . . . ). However, when the interval between the groups is 0.1 to 3 mm or less, the groups may be the same.

The processor 31 specifies each of the groups on the distal side and the proximal side of the lesion as a reference portion (S106). For example, the processor 31 classifies whether the value is equal to or larger than the plaque burden threshold for all the IVUS images according to the determination result, and then specifies each of the groups on the distal side and the proximal side as a reference portion with respect to the lesion. The processor 31 specifies each group positioned on the distal side and the proximal side of the specified lesion among the plurality of grouped reference portions as a reference portion for comparing with the lesion.

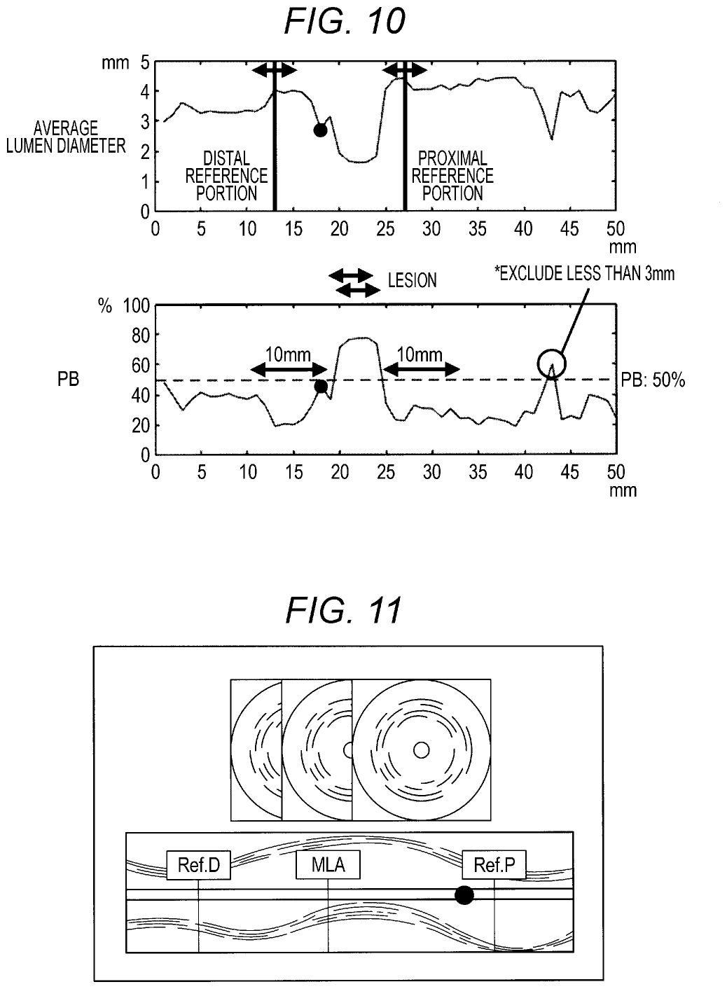

FIG. 10 is a diagram illustrating a display example of information specifying the reference portion. In the display example, a graph of an average lumen diameter and a graph of plaque burden (PB) are displayed side by side vertically. The horizontal axis indicates the length of a blood vessel. When the plaque burden (PB) threshold is 50%, a site exceeding the threshold is specified as a lesion. Portions where the average lumen diameter is maximized at sites within 10 mm on the distal side and the proximal side with respect to the lesion are specified as a distal reference portion and a proximal reference portion, respectively. By displaying such information, it is possible to assist the operator in specifying the reference portion. As illustrated in the present embodiment, the lesion may be a portion having a plaque burden (PB) of 50% or more, for example, and may be a group continuous for 3 mm or more. The reference portion may be a portion having the largest average lumen diameter within 10 mm front of and back of the lesion. When there is a large side branch in the blood vessel and the diameter of the blood vessel greatly changes, the reference portion may be specified between the lesion and the side branch. In specifying the reference portion, the image illustrated in the drawing may be displayed on the display apparatus 4, and correction by the operator may be received. In addition, when the image is displayed on the display apparatus 4, a portion having a large side branch may be presented.

The processor 31 calculates the blood vessel diameters, the lumen diameters, and the areas of the distal and proximal reference portions (S107). The processor 31 calculates the blood vessel diameter (EEM), the lumen diameter, and the area of the reference portion on the distal side and the proximal side. In this case, the length between the reference portions, that is, the length from the distal side) reference portion to the proximal side reference portion may be set to be, for example, 10 mm at the maximum.

The processor 31 controls the input/output I/F 33 to output support information to the display apparatus 4 (S108). As illustrated in the present embodiment as an example, the processor 31 controls the input/output I/F 33 to output support information regarding stent implant to the display apparatus 4 and causes the display apparatus 4 to display the support information. FIG. 11 is a diagram illustrating a display example of information regarding stent implant. In the display example, a transverse tomographic view which is a tomographic view in the axial direction of the blood vessel and a longitudinal tomographic view which is a tomographic view in the radial direction of the blood vessel are displayed side by side vertically. That is, the support information regarding stent implant includes a plurality of longitudinal tomographic views (e.g., cross-sectional view in the radial direction of the blood vessel) by the IVUS image and a transverse tomographic view (e.g., cross-sectional view in the axial direction of the blood vessel) connecting these longitudinal tomographic views. In the transverse tomographic view, the distal reference portion “Ref. D” and the proximal reference portion “Ref. P” are illustrated, and the MLA (minimum lumen area) located between these reference portions is illustrated. By displaying such information, it is possible to assist the operator regarding stent implant.

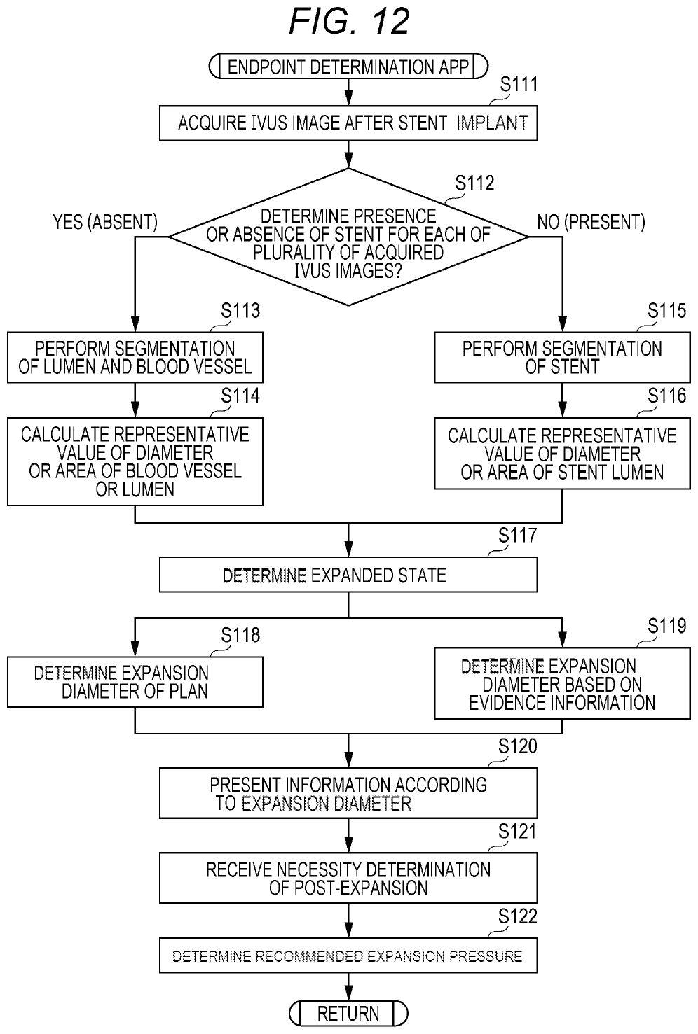

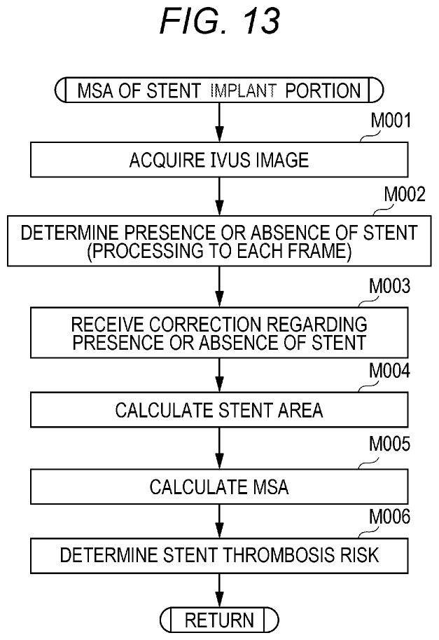

FIG. 12 is a flowchart illustrating an information provision procedure of endpoint determination. FIG. 13 is a flowchart illustrating a processing procedure of MSA calculation. In this flow, provision processing for starting endpoint determination APP to provide support information regarding endpoint determination will be described with reference to FIG. 12.

The processor 31 acquires IVUS images after stent implant (S111). The processor 31 acquires a plurality of IVUS images after stent implant corresponding to one pull back. The processor 31 determines the presence or absence of the stent for each of the plurality of acquired IVUS images (S112). The processor 31 determines the presence or absence of the stent for the plurality of IVUS images by using, for example, the learning model 341 having an object detection function or image analysis processing such as edge detection and pattern patching.

When there is no stent (S112: YES), the processor 31 performs segmentation of the lumen and the blood vessel (Vessel) on the IVUS image without stent (S113). The processor 31 performs segmentation of the lumen and the blood vessel on the IVUS image without a stent, for example, using the learning model 341 having a segmentation function. The processor 31 calculates a representative value of the diameter or the area of the blood vessel or the lumen (S114). The processor 31 calculates the representative value of the diameter or the area based on the segmented lumen and the segmented blood vessel.

When the stent is present (S112: NO), the processor 31 performs segmentation of the stent on the IVUS image with the stent (S115). The processor 31 performs the segmentation of the stent on the IVUS image having the stent, for example, using the learning model 341 having the segmentation function. The processor 31 calculates the representative value of the diameter or area of the stent lumen (S116). The processor 31 calculates the representative value of the diameter or area of the stent lumen based on the segmented stent.

The processor 31 determines the expanded state in the vicinity of the stent implant portion (S117). The processor 31 determines the expanded state in the vicinity of the stent implant portion based on the calculated representative value of the diameter or area of the blood vessel or the lumen and the calculated representative value of the diameter or area of the stent lumen, and causes the display apparatus 4 to display the expanded state. As illustrated in the present embodiment, the expanded state in the vicinity of the stent implant portion displayed on the display apparatus 4 may be, for example, a state in which the range in which the stent is provided is colored and displayed in the transverse tomographic view.

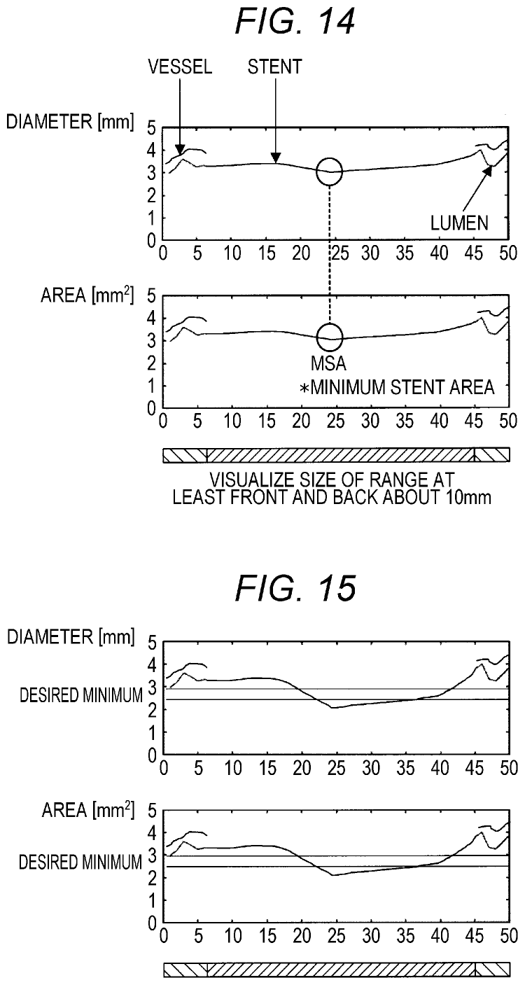

FIG. 14 is a diagram illustrating a visualized display example of the expanded state in the vicinity of the stent implant portion. In the display example, graphical views of the blood vessel, the lumen, and the diameter and area of the stent in a state where the stent lands are displayed side by side vertically. The horizontal axis indicates the length of the blood vessel. In these graphs, the position of the MSA is illustrated. By displaying such information, it is possible to assist the operator to grasp the expanded state in the vicinity of the stent implant portion. In determining the expanded state in the vicinity of the stent implant portion, the processor 31 calculates an MSA in the stent implant portion. Processing of the calculation of the MSA will be described later.

The processor 31 determines the expansion diameter of a plan (S118). For example, the processor 31 refers to a preliminary plan stored in advance in the auxiliary storage unit 34, and determines the expansion diameter of a plan set as desired based on the diameter at the time of stent expansion included in the preliminary plan. The processor 31 may receive an operator's input in determining the expansion diameter of the plan. The processor 31 may generate a graph diagram illustrating the determined desired expansion diameter so as to be superimposed on an image illustrating the expanded state in the vicinity of the stent implant portion.

The processor 31 determines the expansion diameter based on the evidence information (S119). The processor 31 refers to, for example, evidence information such as paper information stored in advance in the auxiliary storage unit 34, and determines a desirable expansion diameter. The processor 31 may receive an input of an operator's own index in determining a desirable expansion diameter. The processor 31 may display a graph illustrating the determined desirable expansion diameter so as to be superimposed on the image illustrating the expanded state in the vicinity of the stent implant portion.

The processor 31 issues information according to the determined expansion diameter (S120). In a case where the determined expansion diameter is equal to or smaller than the desired diameter or area, for example, in a case where the display color is changed, or the like, in a case where the determined expansion diameter is equal to or smaller than the desired diameter or area, and in a case where the determined expansion diameter exceeds the diameter or area, the processor 31 issues information in different display modes. FIG. 15 is a diagram illustrating a display example of information regarding a desired expansion diameter. In the display example, in the blood vessel in a state in which the stent lands, a graphical view of the desired diameter and area of the stent is displayed side by side. The horizontal axis indicates the length of the blood vessel. By displaying such information, it is possible to assist the operator to grasp the desired diameter and area of the stent.

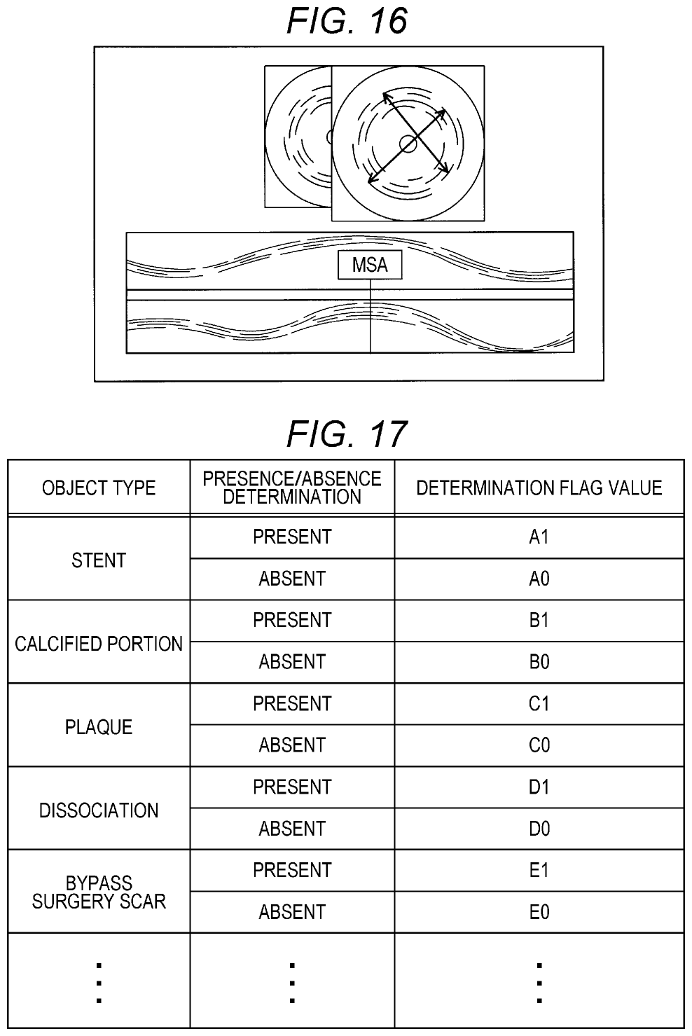

The processor 31 detects an input related to determination of necessity of post-expansion by the operator (S121). The processor 31 determines a recommended expansion pressure based on the expansion diameter at the time of post-expansion (S122). Based on the expansion diameter at the time of post-expansion, the processor 31 refers to the compliance chart stored in the auxiliary storage unit 34, for example, to specify the recommended expansion pressure included in the compliance chart, thereby determining the recommended expansion pressure. The processor 31 causes the display apparatus 4 to superimpose and display the recommended expansion pressure on, for example, an image illustrating an expanded state in the vicinity of the stent implant portion. FIG. 16 is a diagram illustrating a display example of information regarding the endpoint determination. In the display example, a transverse tomographic view which is a tomographic view in the axial direction of the blood vessel and a longitudinal tomographic view which is a tomographic view in the radial direction of the blood vessel are displayed side by side vertically. The transverse tomographic view illustrates the location of the MSA. By displaying such information, it is possible to assist the operator regarding the endpoint determination.

Processing of calculating the minimum stent area (MSA) of the stent implant portion will be described based on FIG. 13. The processing of the MSA calculation may be performed, for example, as subroutine processing in the processing of S117 for determining the expanded state in the vicinity of the stent implant portion.

The processor 31 acquires IVUS images (M001). The processor 31 acquires the IVUS images corresponding to one pull back. The processor 31 determines the presence or absence of a stent (M002). The processor 31 determines the presence or absence of the stent in the frame of each IVUS image, and stores the processing result for each frame in, for example, an array (i.e., sequence type variable).

The processor 31 acquires information about a correction regarding the presence or absence of a stent based on an operation by the operator (M003). The processor 31 specifies the stent area by performing processing on the frame of each IVUS image including the stent. In performing the processing, the processor 31 may calculate the diameter and area of the lumen using the learning model 341 having the segmentation function. Alternatively, the processor 31 may calculate a minor axis and a major axis in the lumen diameter and determine an eccentricity (i.e., minor axis/major axis) by dividing the minor axis by the major axis.

The processor 31 calculates the MSA (M005). The processor 31 calculates the MSA based on the calculated lumen diameter, area, and eccentricity in the specified stent area. The processor 31 determines a stent thrombosis risk (M006). For example, the processor 31 may function as an MSA determination device, determine whether the size is larger than 5.5 square mm (MSA>5.5 [mm2]), and determine that there is no stent thrombosis risk when the size is larger than 5.5 square mm.

According to the present embodiment, the image processing apparatus 3 generate object information regarding the presence or absence and the type of an object based on the medical images such as IVUS images acquired using the image diagnosis catheter 1. Since the image processing apparatus 3 performs the provision processing for providing the support information to the operator of the image diagnosis catheter 1 such as a physician based on the object information, it is possible to provide the operator with appropriate support information according to the presence or absence and the type of the object included in the medical image generated using the image diagnosis catheter 1.

According to the present embodiment, the image processing apparatus 3 inputs a medical image to the learning model 341, and generates object information by using the type of object estimated by the learning model 341. Since the learning model 341 is trained to estimate the object included in the medical image by inputting the medical image, the image processing apparatus 3 can efficiently acquire the presence or absence of the object included in the medical image and the type of the object in a case where the object is included.

According to the present embodiment, since the type of an object specified as being included in a medical image includes at least one of an epicardium, a side branch, a vein, a guide wire, a stent, a plaque deviating into a stent, a lipid plaque, a fibrous plaque, a calcified portion, blood vessel dissociation, thrombus, and a blood type, appropriate support information can be provided to the operator according to the object that can be the region of interest such as the lesion in the luminal organ.

According to the present embodiment, since the provision processing of the support information performed according to the object information includes the provision processing for providing the support information regarding the stent implant and the endpoint determination, in a case where the type of the target object is the stent, appropriate support information according to the presence or absence of the stent can be provided to the operator.

### Second Embodiment

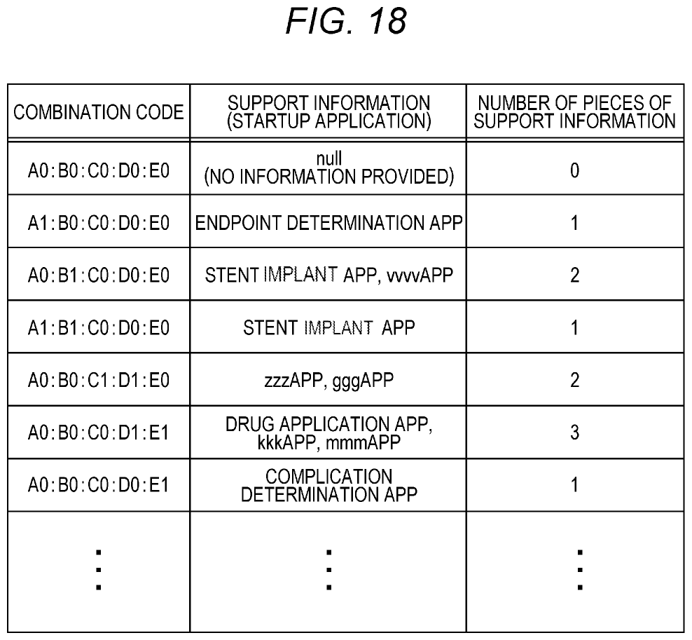

FIG. 17 is a diagram illustrating an example of a relation table in a second embodiment. FIG. 18 is a diagram illustrating an example of a combination table according to the second embodiment. Similarly to the first embodiment, the relation table in the second embodiment includes, for example, an object type and presence/absence determination as management items or fields of the relation table, and further includes a determination flag value.

In the management item of the object type, the type of the object such as the stent is stored as in the first embodiment. In the management item of the presence/absence determination, the presence/absence of each object type is stored as in the first embodiment.

In the management item of the determination flag value, a determination flag value according to the presence or absence of the object type stored in the same record is stored. As an example, the determination flag value includes a type flag indicating the object type and a presence/absence flag indicating the presence or absence of the object, and is configured by a value in which the type flag and the presence/absence flag are connected. In the present embodiment, alphabets such as “A” and “B” indicate the corresponding type of each object (e.g., stent and a calcified portion), and numbers of 1 and 0 indicate the presence or absence of the object. By using such a determination flag value, it is possible to uniquely determine a value indicating presence or absence in each object type.

The combination table includes, for example, a combination code, support information, and the number of pieces of support information as management items of the table. In the management item of the combination code, for example, information indicating a combination of determination flag values indicated in the relation table is stored.

The combination code includes a character string in which determination flag values indicating presence (i.e., “1”) or absence (i.e., “0”) in each object type are concatenated. For example, when the combination code is “A0:B0:C0:D0:E0,” it indicates that there are not all objects indicated by “A” to “E” in the IVUS image. When the combination code is “A1:B0:C0:D0:E0,” it indicates that there is only an object of A (i.e., stent). When the combination code is “A1:B1:C0:D0:E0,” it indicates that there are only “A” (i.e., stent) and “B” (i.e., calcified portion). As described above, even in a case where a plurality of types of objects is included in the IVUS image, it is possible to uniquely determine the value indicating the combination of the presence and absence in each object type by using the combination code.

In the management item of the support information, for example, the content of the support information according to the combination code stored in the same record or the application name for providing the support information is stored. The stored support information is not limited to one, and may be two or more. Alternatively, depending on the combination code, information indicating that there is no stored support information may be stored. For example, in a case where the combination code is “A0:B0:C0:D0:E0,” it can be said that any type of object is not included in the IVUS image and the blood vessel illustrated in the IVUS image is healthy, and the processor 31 may not perform the processing for providing the support information. For example, in a case where the combination code is “A0:B0:C0:D1:E1,” it is indicated that the IVUS image includes a plurality of objects, and a plurality of pieces of support information corresponding to the plurality of objects may be stored.

The processor 31 may perform provision processing (i.e., execution of a startup application) on all of the plurality of pieces of support information. Alternatively, the processor 31 may determine a selection of any one of the plurality of pieces of support information and perform the provision processing of the selected support information. For example, by generating object information according to the format of the combination code, the processor 31 can compare the object information with the combination table to efficiently determine the support information.

In the management item of the number of pieces of support information, for example, the number of pieces of support information stored in the same record is stored. The processor 31 may change the display mode at the time of executing the provision processing of the support information according to the number stored in the management item of the number of pieces of support information.

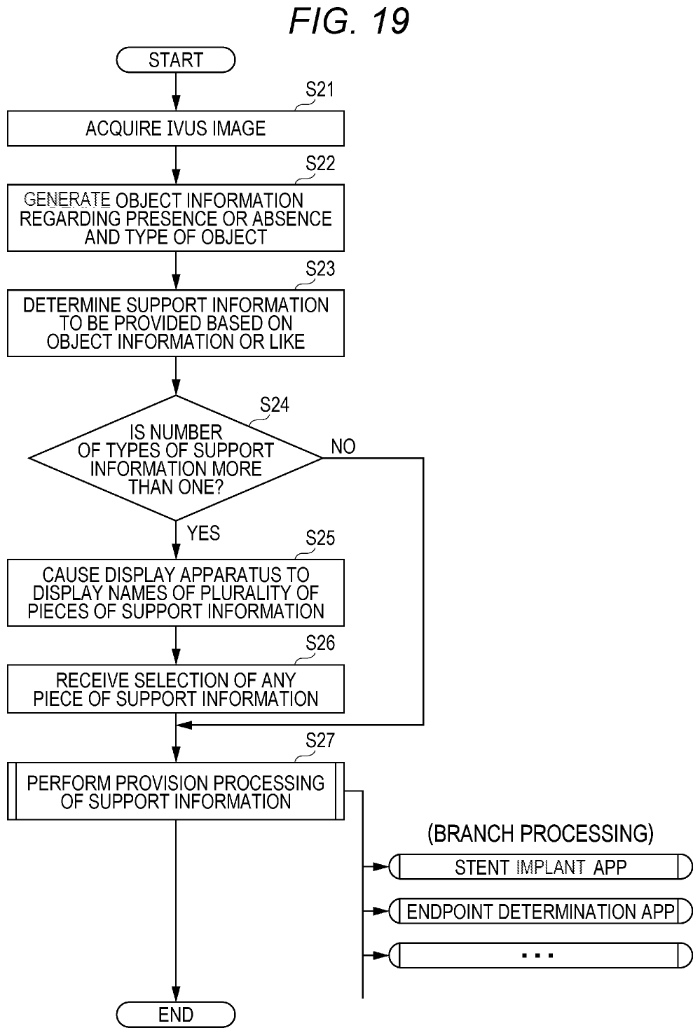

FIG. 19 is a flowchart illustrating information processing performed by the processor 31. The processor 31 of the image processing apparatus 3 executes the following processing based on input data or the like output from the input apparatus 5 in response to an operation of an operator of the image diagnosis catheter 1 such as a physician.

The processor 31 acquires IVUS images (S21). The processor 31 generates object information regarding the presence or absence and the type of an object included in the IVUS image (S22). Similarly to S11 to S12 of the first embodiment, the processor 31 performs processing from S21 to S22.

The processor 31 determines the support information to be provided based on the object information and the like (S23). For example, the processor 31 may refer to the relation table stored in the auxiliary storage unit 34 to generate the object information based on the presence or absence of all types of objects defined in the relation table. The learning model 341 has trained about all types of objects, and by inputting the IVUS image to the learning model 341, the processor 31 can acquire the presence or absence of all types of objects defined in the relation table. The processor 31 compares the object information with, for example, the combination table stored in the auxiliary storage unit 34 to determine the support information to be provided, thereby specifying the number of pieces of the support information.

The processor 31 determines whether the number of types of the support information to be provided is plural (S24). The processor 31 determines whether the number of types of support information determined according to the object information is plural, for example, by referring to the combination table stored in the auxiliary storage unit 34.

When there are a plurality of types of support information to be provided (S24: YES), the processor 31 causes the display apparatus 4 to display the names of the plurality of pieces of support information (S25). The processor 31 determines a selection of any piece of support information (S26). The processor 31 causes the display apparatus 4 to display the names and the like of the plurality of pieces of support information in the form of a list, for example, and determines any support information selected by the user according to a touch panel function included in the display apparatus 4 or an operation of the user by the input apparatus 5.

The processor 31 performs the provision processing of the support information (S27). After the processing of S26 or in a case where the number of types of the support information to be provided is not plural (S24: NO), the processor 31 performs the provision processing of the support information. When the processing of S26 is performed, the processor 31 performs the provision processing of the support information selected in the processing of S26. In a case where the number of types of the support information to be provided is not plural (S24: NO), that is, in a case where the number of types of the support information to be provided is a single type, the processor 31 performs the provision processing of the support information determined in the processing of S23. The processor 31 performs the provision processing of the support information such as the stent implant APP or the endpoint determination APP on the selected or determined support information as in the first embodiment.

According to the present embodiment, the relation table in which the type of each object and the corresponding support information are associated with each other is stored, for example, in a predetermined storage area accessible by the processor 31 of the image processing apparatus 3, such as the storage unit. Therefore, the processor 31 can efficiently determine the support information according to the type of the object by referring to the relation table stored in the storage unit. The relation table includes not only the support information according to the presence or absence of a specific type of object but also support information according to a combination of the presence or absence of each of a plurality of types of objects. Therefore, appropriate support information can be provided to the operator according to not only the presence or absence of a single type of object but also the combination of the presence or absence of each of a plurality of types of objects.

It should be understood that the embodiments disclosed herein are illustrative in all respects and are not restrictive. The technical features described in the examples can be combined with each other, and the scope of the present invention is intended to include all modifications within the scope of the claims and the scope equivalent to the claims.

What is claimed is:

1. A medical system comprising:
a catheter that includes a sensor and can be inserted into a luminal organ;
a display apparatus; and
an image processing apparatus configured to:
store a plurality of pieces of support information each related to a medical operation or diagnosis on the luminal organ and associated with a type of an object,
generate an image of the luminal organ based on a signal output from the sensor of the catheter,
input the generated image to a machine learning model and acquire an output indicating a type of an object that is present in the image,
acquire input information indicating a medical operation or diagnosis to be performed,
determine one of the pieces of support information corresponding to the type of the object and the medical operation or diagnosis indicated by the input information, and
cause the display apparatus to display said one of the pieces of support information.

2. The medical system according to claim 1, wherein the image processing apparatus is configured to:
store a plurality of application programs corresponding to the plurality of pieces of support information, and
execute a corresponding one of the application programs for displaying said one of the pieces of support information.

3. The medical system according to claim 1, wherein the type of the object includes at least one of: an epicardium, a side branch, a vein, a guide wire, a stent, a plaque deviating into a stent, a lipid plaque, a fibrous plaque, a calcified portion, blood vessel dissociation, thrombus, and a blood type.

4. The medical system according to claim 1, wherein
the medical operation or diagnosis is related to a stent placed in the luminal organ, and
the image processing apparatus is configured to:
determine whether the stent is present in the image,
in response to determining that the stent is present, determining support information regarding endpoint determination as said one of the pieces of support information to be displayed, and
in response to determining that the stent is not present, determining support information regarding stent implant as said one of the pieces of support information to be displayed.

5. The medical system according to claim 1, wherein the image processing apparatus cause the display apparatus to display the image in association with said one of the pieces of support information.

6. The medical system according to claim 1, wherein the image processing apparatus stores a table in which each of the plurality of pieces of support information is associated with the corresponding type of the object.

7. The medical system according to claim 1, wherein the image processing apparatus is configured to:
cause the display apparatus to display a plurality of candidates of support information corresponding to the type of the object and the medical operation or diagnosis indicated by the input information, and
receive a selection of one of the candidates of support information as said one of the pieces of support information to be displayed.

8. The medical system according to claim 1, wherein the luminal organ is a blood vessel.

9. The medical system according to claim 8, wherein
the sensor of the catheter includes an ultrasound transmitter and receiver, and
the image processing apparatus is configured to generate an ultrasonic tomographic image of the blood vessel based on a signal that is output from the sensor.

10. The medical system according to claim 8, wherein
the sensor of the catheter includes an optical transmitter and receiver, and
the image processing apparatus is configured to generate an optical coherence tomographic image of the blood vessel based on a signal that is output from the sensor.

11. A method for processing a medical image of a luminal organ, comprising:
storing a plurality of pieces of support information each related to a medical operation or diagnosis on the luminal organ and associated with a type of an object;
generating an image of the luminal organ based on a signal from a sensor of a catheter inserted into the luminal organ;
inputting the generated image to a machine learning model and acquiring an output indicating a type of an object that is present in the image;
receiving an input of information indicating a medical operation or diagnosis to be performed;
determining one of the pieces of support information corresponding to the type of the object and the medical operation or diagnosis indicated by the input information; and
displaying said one of the pieces of support information.

12. The method according to claim 11, wherein displaying includes starting an application for displaying said one of the pieces of support information.

13. The method according to claim 11, wherein the type of the object includes at least one of: an epicardium, a side branch, a vein, a guide wire, a stent, a plaque deviating into a stent, a lipid plaque, a fibrous plaque, a calcified portion, blood vessel dissociation, thrombus, and a blood type.

14. The method according to claim 11, wherein
the medical operation or diagnosis is related to a stent placed in the luminal organ,
the method further comprises determining whether the stent is present in the image, and
determining one of the pieces of support information includes:
in response to determining that the stent is present, determining support information regarding endpoint determination as said one of the pieces of support information to be displayed, and
in response to determining that the stent is not present, determining support information regarding stent implant as said one of the pieces of support information to be displayed.

15. The method according to claim 11, wherein displaying includes displaying the image in association with said one of the pieces of support information.

16. The method according to claim 11, wherein storing includes storing a table that associates each of the pieces of support information with the corresponding type of the object.

17. The method according to claim 11, wherein determining one of the pieces of support information includes:
displaying a plurality of candidates of support information corresponding to the determined type of the object and the medical operation or diagnosis indicated by the input information, and
receiving a selection of one of the candidates of support information as said one of the pieces of support information to be displayed.

18. The method according to claim 11, wherein the luminal organ is a blood vessel.

19. The method according to claim 18, wherein the generated image is an ultrasonic tomographic image or an optical coherence tomographic image of the blood vessel.

20. A medical image processing apparatus comprising:
a memory that stores a plurality of pieces of support information each related to a medical operation or diagnosis on a luminal organ and associated with a type of an object;
an interface circuit connectable to a display apparatus and a catheter that includes a sensor and can be inserted into a luminal organ; and
a processor configured to:
generate an image of the luminal organ based on a signal output from the sensor of the catheter,
input the generated image to a machine learning model and acquire an output indicating a type of an object that is present in the image,
acquire input information indicating a medical operation or diagnosis to be performed,
determine one of the pieces of support information corresponding to the type of the object and the medical operation or diagnosis indicated by the input information, and
cause the display apparatus to display said one of the pieces of support information.
